(19) United States
(12) Patent Application Publication
(10) Pub. No.: US 2025/0107717 A1
(43) Pub. Date: Apr. 3, 2025

(54) CALIBRATION-FREE CUFFLESS BLOOD PRESSURE MONITORING SYSTEM AND DEVICE

(71) Applicants: Pallob Kumar Gain, Khulna (BD); Md Jahirul Islam, Khulna (BD); Brian Carlson, Lynchburg, VA (US)

(72) Inventors: Pallob Kumar Gain, Khulna (BD); Md Jahirul Islam, Khulna (BD); Brian Carlson, Lynchburg, VA (US)

(21) Appl. No.: 18/900,728

(22) Filed: Sep. 28, 2024

Related U.S. Application Data
(60) Provisional application No. 63/586,055, filed on Sep. 28, 2023.

(51) Int. Cl. A61B 5/021; A61B 5/024; A61B 5/0245; A61B 5/26; A61B 5/282; A61B 5/332; A61B 5/339

(52) U.S. Cl. CPC A61B 5/02108; A61B 5/02416; A61B 5/02438; A61B 5/0245; A61B 5/26; A61B 5/282; A61B 5/332; A61B 5/339

(57) ABSTRACT

The present invention is a self-contained (no wires or external probes), cuffless, handheld blood pressure monitor that does not require calibration and provides results that are equivalent to a traditional cuff-based blood pressure measuring technique. It achieves this result by using various mathematical relationships between pulse wave velocity, age, height, and weight to calculate a user's SBP and DBP. Consequently, this compact device allows for a user to easily track their blood pressure throughout the course of a day/week/year without having to visit a medical office.

### RELATED APPLICATION

This application claims priority to U.S. Provisional Patent Application Ser. No. 63/586,055 for “Calibration-Free Cuffless Blood Pressure Monitoring System and Device” filed Sep. 28, 2023, and currently co-pending, the entirety of which is incorporated herein by reference.

### FIELD OF THE INVENTION

The present invention pertains generally to a handheld device that allows for the cuffless measurement of blood pressure.

### BACKGROUND OF THE INVENTION

Almost half of the American population over the age of 20 have high blood pressure. Currently, the conventional way to measure blood pressure is at a doctor's office with a cuff that is attached to a blood pressure monitor. While this method is typically more accurate than other home-based methods, it comes with a set of serious drawbacks.

The first drawback is that if a patient wants to track their blood pressure they typically have to travel to a medical office, which can take up a significant amount of time. Even if the patient decides that they want to track their blood pressure at their place of residence, then they often need to buy a small cuff-based machine which are often inaccurate, bulky and difficult to travel with.

The cuff itself for cuffed blood pressure monitors has its own set of issues. The cuff needs to be wrapped around a user's arm prior to having a reading performed with a blood pressure monitor. This is a task that not everyone can do on their own, thus creating a need for a second person and making the process more cumbersome than needed. Additionally, a cuff can only be used a limited number of times per day because it squeezes down on a user's arm during measurement. If a user was to use the cuffed blood pressure monitor more often than what is recommended, then they risk damaging the brachial artery that runs through the arm.

There are other cuffless blood pressure monitors available that attempt to solve many of the problems outlined above, but they have their own set of issues. The biggest of which is that these cuffless devices are not as accurate as their cuffed counterpart. Consequently, a user who is tracking their blood pressure may not feel comfortable relying on the numbers that are displayed by the currently available models. In addition, they also need to be calibrated against a previously taken cuff-based reading.

There is a need in the art for a cuffless blood pressure monitor that makes the process of tracking blood pressure much more convenient for a user, but without compromising the accuracy of a reading.

### SUMMARY OF THE INVENTION

The invention disclosed in this application is generally directed towards a handheld device that accurately and reliably tracks a user's blood pressure through a cuffless method. It was specifically designed to be a compact device that can be easily stored in either a pocket or purse so that it can be transported from location to location. Additionally, the compact design will also let a user easily track their blood pressure throughout the day in a variety of different settings and without any of the use restrictions that are common with cuffed blood pressure monitors. Finally, the present invention does not need to be calibrated like other blood pressure monitors that are currently available in the market.

The present invention achieves this based on the relationship between pulse wave velocity and blood pressure. Typically, pulse wave velocity is used to measure the arterial stiffness of a patient; however, it can also be used to track blood pressure. When the heart contracts to pump blood, there is a pressure wave that is generated inside the arteries that then proceeds to travel throughout the body. There are two different types of pulse wave velocities that can be measured by the present invention; the first pulse wave velocity corresponds with systolic blood pressure, and the second corresponds with diastolic blood pressure. Further, it is possible to calculate a mean arterial pressure based on a user's systolic and diastolic blood pressure.

The present invention works by analyzing heart activity from two different sources; one is based on an electrocardiogram (ECG) reading through the use of two electrodes, and the second is based on a photoplethysmography (PPG) reading through the use of an infrared optical sensor. Next, the present invention will take the results of those two readings and data entered from a user to perform a series of calculations in order to accurately provide a blood pressure reading. The accuracy and reliability of these results are enhanced by sending both the ECG and PPG signals through a second order digital signal processing filter.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a front perspective view of the handheld blood pressure monitor;

FIG. 2 is a back perspective view of the handheld blood pressure monitor;

FIG. 3 is a front perspective view of the handheld blood pressure monitor while in use;

FIG. 4 is a front view of the handheld blood pressure monitor while connected to a computer;

FIG. 5 is a graph showing the results of an ECG and PPG test measured by the cuffless blood pressure monitor overlayed onto each other;

FIG. 6 is a flow chart of the system operation for the cuffless blood pressure monitor;

FIG. 7 shows a prototype of the user interface that will be displayed on the cuffless blood pressure monitor;

FIG. 8 is an alternative embodiment of the cuffless blood pressure monitor installed into a weight scale; and

FIG. 9 is an alternative embodiment of a compact version of the cuffless blood pressure monitor that can be easily attached and removed from the USB port of a smart phone or tablet.

### DETAILED DESCRIPTION

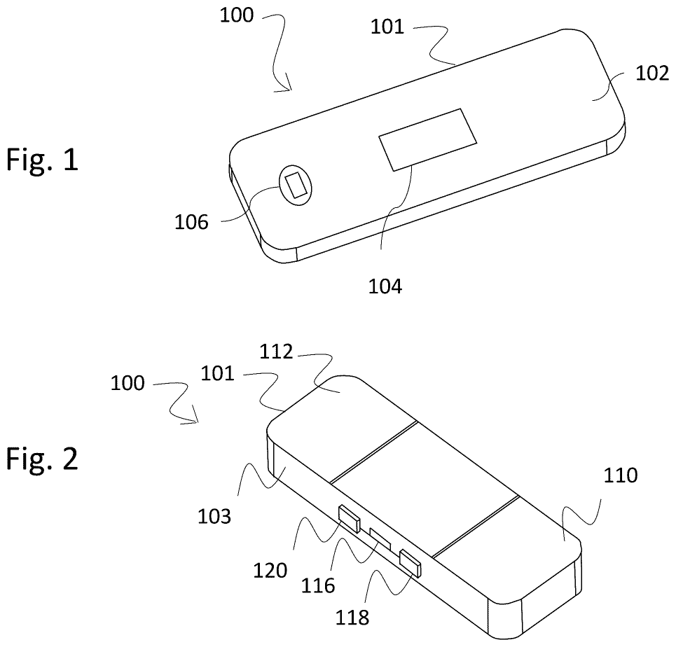

Referring initially to FIGS. 1 to 3, a front perspective view, a back perspective view, and a front view of the cuffless blood pressure monitor in use is shown and generally designated as system 100.

FIG. 1 shows the front perspective view of cuffless blood pressure monitor 100 having a housing unit 101 that holds all of the different components that make up the device. Front surface 102 has display 104 located in the middle of the device, and photoplethysmography (PPG) sensor 106 located on the left side. The placement of PPG sensor 106 was intentional so that a user can easily cover it with their thumb 107. In this embodiment PPG sensor 106 will be a reflective infrared optical sensor, but other sensors known in the art that achieve a similar result are fully contemplated.

In the present embodiment, display 104 is configured to display a user's heart rate, a mean arterial pressure (MAP) value, and indicate whether the MAP is high, low, or normal. In other embodiments, display 104 will also be able to show current readings for a user's pulse, systolic blood pressure (SBP), and diastolic blood pressure (DBP). Additionally, display 104 will be a small OLED screen that is sized to fit within the device. However, other displays that are currently available in the art are currently envisioned.

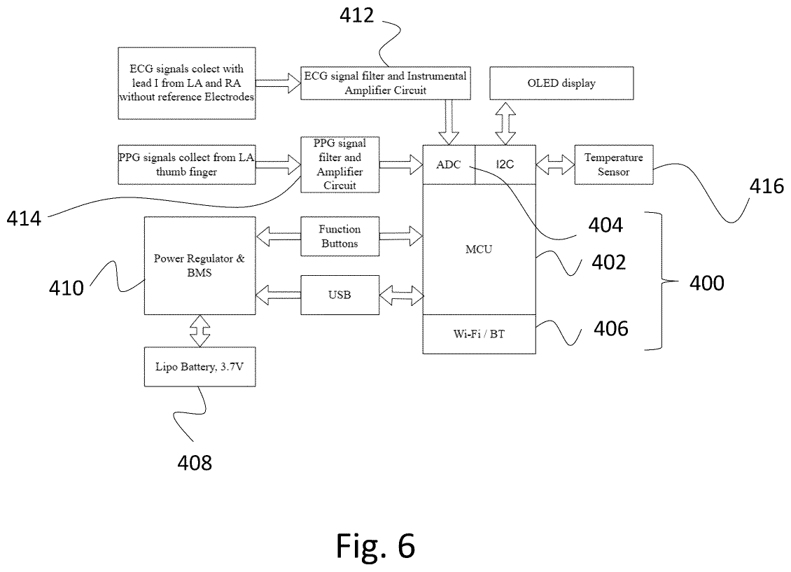

Internally, housing unit 101 houses control system 400 (discussed in detail for FIG. 6) that is responsible for performing all of the different calculations that cuffless blood pressure monitor 100 will perform. Critically, control system 400 will be in electrical communication with all of the different sensors that make up cuffless blood pressure monitor 100.

FIG. 2 shows the back perspective view of cuffless blood pressure monitor 100. ECG right electrode 110 and ECG left electrode 112 are installed onto back surface 110. When a user decides to get a blood pressure reading, they will be required to place their fingers from both their right and left hand onto the respective sensors. The two different electrodes will then sense the ECG signal and heart rate required for cuffless blood pressure monitor 100 to determine a user's heart rate.

Also shown in this view is top surface 103 where power button 120, USB Port 116, and function button 118 are shown. Power button 120 is responsible for turning the cuffless blood pressure monitor on and off. USB Port 116 can be sized for either a micro-USB connection or a USB-C connection, which allows cuffless blood pressure monitor to either charge, or be plugged into a user's computer for synchronization purposes. Function button 118 allows a user to input user parameters (Weight, Height, Age) and cycle through the various functions that cuffless blood pressure monitor 100 is able to perform. The main function will be the tracking of blood pressure, but a user will also be able to independently track their heart rate in a separate function.

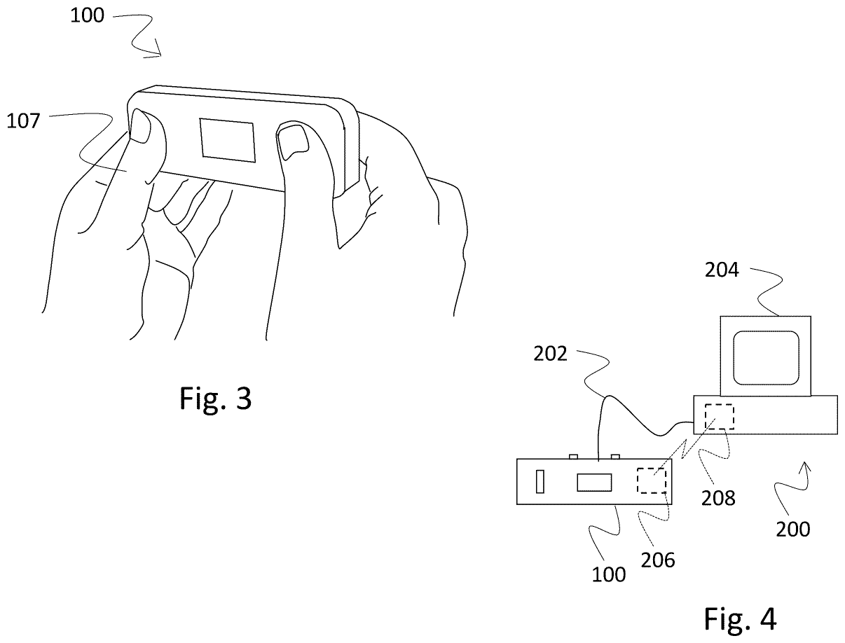

FIG. 3 shows a user holding the device and having their blood pressure monitored. This view shows the user's left thumb covering PPG sensor 106 on front surface 102, their left and right fingers resting atop ECG right electrode 110 and ECG left electrode 112 on back surface 108. The compact design of cuffless blood pressure monitor 100 will allow a user to use the device in a wide variety of different environments. Additionally, the compact design easily allows a user to transport cuffless blood pressure monitor 100 from location to location within either a pocket, personal bag, or purse.

Referring now to FIG. 4, an image with cuffless blood pressure monitor 100 connected to a user's computer 200 via a micro-USB cable 202 is shown. Connected to computer 200 is monitor 204 to display the companion program for cuffless blood pressure monitor 100. The companion program allows a user to enter inputs that will later be used by cuffless blood pressure monitor 100 to calculate a user's MAP. In a non-limiting example, the variables entered by a user include age, weight, height, and gender. When cuffless blood pressure monitor 100 is connected to computer 200, these variables will then be uploaded to the device via micro-USB 202. Additionally, a user will be able to upload measurements taken throughout a given period of time to computer 200.

In another embodiment, cuffless blood pressure monitor 100 has Bluetooth adapter 206 (shown in dashed lines) built into it to communicate wirelessly with the corresponding Bluetooth receiver 208 (shown in dashed lines) in computer 200. This will provide cuffless blood pressure monitor 100 with the ability to wirelessly synchronize with computer 200, or additionally with a smart phone or smart tablet.

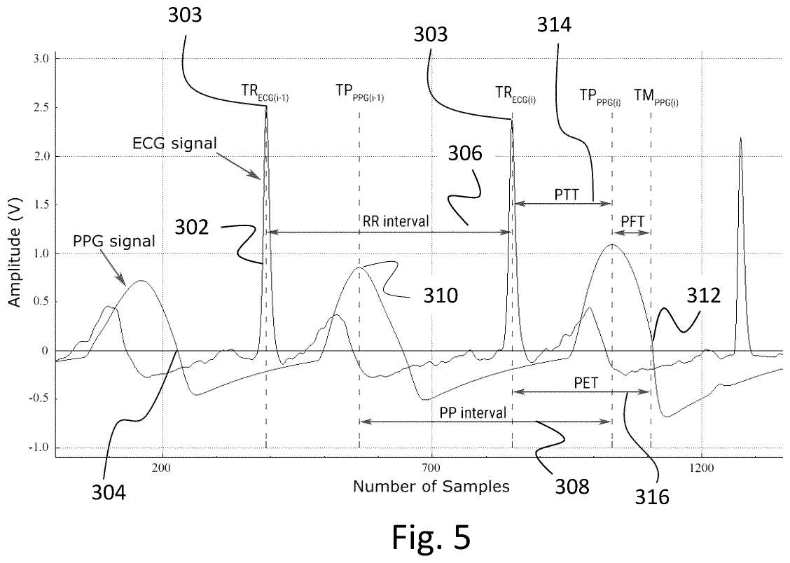

Referring now to FIG. 5, a graph of collected ECG and PPG data overlayed onto each is shown. This graph details what is measured by cuffless blood pressure monitor 100 in order to calculate a user's blood pressure in conjunction with the data that is entered by a user. While the subsequent steps are discussed in a sequential order, this was done merely to describe the general process behind the operation of cuffless blood pressure monitor 100 and was not intended to be a limiting disclosure. The following steps can be performed in any given order, or even simultaneously of one another.

The first step in the process is for cuffless blood pressure monitor 100 to determine both ECG signal 302 and PPG signal 304. ECG signal 302 for a user is measured by right electrode 110 and left electrode 112. Once the ECG signal for a user is determined, then cuffless blood pressure monitor 100 will measure RR interval 306, which is the measured time difference between the different R peaks for the ECG, these R peaks are shown as TR_{ECG} 303 on the graph. Cuffless blood pressure monitor 100 will then determine TR_{ECG} 303 based on RR interval 306 so that it can calculate the heart rate based on ECG signal 302 with the equation HR_{ECG}(i)=60/(TR_{ECG(i)}−TR_{ECG(i-1)}).

Cuffless blood pressure monitor 100 then determines PPG signal 304 based on the readings that are collected by PPG sensor 106. Once PPG signal 304 is measured, cuffless blood pressure monitor 100 will then measure TP_{PPG} 310 which is calculated by measuring PP interval 308. Once TP_{PPG} is measured cuffless blood pressure monitor 100 will then calculate the PPG heart rate with the equation: HR_{PPG(i)}=60/(TP_{PPG(i)}−TP_{PPG(i-1)}).

A crucial feature of this first step is that cuffless blood pressure monitor 100 will both filter and amplify the different measurements taken to determine ECG signal 302 and PPG signal 304. This filtering process drastically improves the accuracy and reliability of the Systolic and Diastolic Blood Pressure readings that are eventually calculated and displayed by cuffless blood pressure monitor 100. Additionally, cuffless blood pressure monitor 100 will check to ensure that two different measured heart rates are within an acceptable range, which further improves the accuracy of the overall system. Cuffless blood pressure monitor 100 will calculate this error rate with the equation HRₑᵣᵣₒᵣ₍ᵢ₎=|HR_{PPG(i)}−HR_{ECG(i)}|. In addition to ensuring that HRₑᵣᵣₒᵣ₍ᵢ₎ is within an acceptable range, cuffless blood pressure monitor 100 will also calculate the average for the two different heart rates determined from ECG signal 302 and PPG signal 304. The equation that cuffless blood pressure monitor 100 will use is HRᵢ=HR_{ECG(i)}+HR_{PPG(i)}/2.

The next series of steps for cuffless blood pressure monitor 100 will be to determine the Pulse Transit Time (PTT) 314 and Pulse Elimination Time (PET) 316. PTT 314 is the amount of time it takes the pressure wave from a heartbeat to travel from the heart to the PPG sensor 106. Cuffless blood pressure monitor 100 calculates PTT 314 by measuring the time delay between TP_{PPG} 310 of PPG signal 304 and TR_{ECG} 303 of ECG signal 302 in the same cardiac cycle (PTT₍ᵢ₎=TP_{PPG(i)}−TR_{ECG(i)}). It has been realized through the research efforts that went into the development of cuffless blood pressure monitor 100 that systolic blood pressure is highly related to PTT.

With PTT determined, cuffless blood pressure monitor 100 moves on to calculating PET 316. PET 316 is the amount of time it takes for blood to leave a user's thumb that is resting atop PPG sensor 106. Cuffless blood pressure monitor 100 will determine PET by measuring the time delay between the 50% down stroke (shown as point 312) of the PPG signal 304 and TR_{ECG} 303 of ECG signal 302 in the same cardiac cycle (PET₍ᵢ₎=TM_{PPG(i)}−TR_{ECG(i)}). It has been realized through the research efforts that went into the development of cuffless blood pressure monitor 100 that diastolic blood pressure is highly related to PET.

With both the PTT and PET values calculated, cuffless blood pressure monitor 100 can then move on to determining the different pulse wave velocity values. Cuffless blood pressure monitor 100 will perform calculations to determine two different pulse wave velocities; one that corresponds with systolic blood pressure, and another that corresponds with diastolic blood pressure. The pulse wave velocity for systolic blood pressure can be defined as Pulse Transit Velocity (PTV), and the pulse wave velocity for diastolic blood pressure can be defined as Pulse Eliminate Velocity (PEV). A key contributor to these relationships is the Pulse Travel Distance (PTD). PTD is a key component of the calculations because it allows cuffless blood pressure monitor 100 to take into account the height of each user; both of which impact of how far a pulse wave must travel. This means that cuffless blood pressure monitor 100 is able to calculate a unique PTV and PEV for each user.

The PTD is the distance between the exit point of blood leaving the heart and reaching the terminal of the left-hand thumb finger. Generally, this can be determined based on an individual's wingspan which is typically the same length as an individual's height. However, this is not always the most accurate way to determine the PTD because frequently an individual's wingspan can be longer than their height. The body distance factor (BDF) is a crucial factor that cuffless blood pressure monitor 100 uses to address this shortcoming. BDF was a factor that was independently determined during the development of cuffless blood pressure monitor 100 and ranges anywhere from 0.49-0.51 depending on the individual. Multiplying the BDF by a user's height provides a much more accurate measurement of PTD and subsequently allows for a much more accurate calculation of PTV and PEV.

$$PTD = SUBJECT\_HEIGHT \times BODY\_DISTANCE \_FACTOR .$$

$${PTV}_{( i )} = \frac{PTD}{{PTT}_{( i )}}$$

$${PEV}_{( i )} = \frac{PTD}{{PET}_{( i )}} .$$

Now that cuffless blood pressure monitor 100 has values for PTV₍ᵢ₎, PEV₍ᵢ₎, and HR₍ᵢ₎ it will then move to store each of these values in three separate arrays. Once cuffless blood pressure monitor 100 has five entries in each array, the system will check the stability of the data collected by applying a standard deviation low-pass autocorrelation algorithm. To do so, cuffless blood pressure monitor 100 will determine the average PTV,

$$\overline{PTV} = \frac{{\sum }_{i = 0}^{N - 1} {PTV}_{( i )}}{N} ,$$

and then the subsequent standard deviation,

$${S}_{x} = \sqrt{\frac{{\sum }_{i = 0}^{N - 1} {( {PTV}_{( i )} - \overline{PTV} )}^{2}}{N - 1}} ,$$

to ensure that there is an acceptable variance for the collected data. If cuffless blood pressure monitor 100 determines that there is not an acceptable variance, then it will start the entire process over again and do so until the data is within an acceptable variance.

Once the data set is accepted, the system evaluates the individual readings within the five readings in the PTV sequence (Auto Correlation). If there are outliers within the five readings, they are also removed from the data set. Here, Autocorrelation |r(k)|<CORRELATION_THRESHOLD. This is calculated using the given formula below;

$$r ( k ) = \frac{{\sum }_{i = 0}^{N - 1} ( {PTV}_{( k )} - \overline{PTV} ) \times ( {PTV}_{( i )} - \overline{PTV} ) ; for i \neq k}{( N - 1 ) \times {S}_{x}^{2}}$$

This provides the most accurate values for HRₖ, PTV₍ₖ₎, and PEV₍ₖ₎. A new P̅T̅V̅, P̅E̅V̅, and H̅R̅ is calculated with outlying data sets (if any) are removed.

The final step before the values for SBP, DBP, and MAP can be determined is for cuffless blood pressure monitor 100 to calculate a user's BMI. The BMI is determined by the standard BMI formula and is dependent on a user's entered weight and height.

Once cuffless blood pressure monitor 100 determines that all of the individual data points have an acceptable correlation threshold, then the different values for Systolic Blood Pressure (SBP), Diastolic Blood Pressure (DBP), and Mean Arterial Pressure (MAP) can be calculated.

$$SBP = {a}_{s} + {b}_{s} \times \overline{PTV} + {c}_{s} \times BMI + {d}_{s} \times Age + {e}_{s} \times {\overline{PTV}}^{2} + {f}_{s} \times {BMI}^{2} + {g}_{s} \times {Age}^{2} + {h}_{s} \times {\overline{{PTV}^{}}}^{3} + {i}_{s} \times {BMI}^{3} + {j}_{s} \times {Age}^{3} + {k}_{s} \times \overline{PTV} \times BMI + {l}_{s} \times BMI \times Age + {m}_{s} \times Age \times \overline{PTV} + {n}_{s} \times \overline{PTV} \times BMI \times Age + {o}_{s} \times {\overline{PTV}}^{2} \times BMI \times Age + {p}_{s} \times \overline{PTV} \times {BMI}^{2} \times Age + {q}_{s} \times \overline{PTV} \times BMI \times {Age}^{2}$$

$$DBP = {a}_{d} + {b}_{d} \times \overline{PEV} + {c}_{d} \times BMI + {d}_{d} \times Age + {e}_{d} \times {\overline{PEV}}^{2} + {f}_{d} \times {BMI}^{2} + {g}_{d} \times {Age}^{2} + {h}_{d} \times {\overline{{PEV}^{}}}^{3} + {i}_{d} \times {BMI}^{3} + {j}_{d} \times {Age}^{3} + {k}_{d} \times \overline{PEV} \times BMI + {l}_{d} \times BMI \times Age + {m}_{d} \times Age \times \overline{PEV} + {n}_{d} \times \overline{PEV} \times BMI \times Age + {o}_{d} \times {\overline{PEV}}^{2} \times BMI \times Age + {p}_{d} \times \overline{PEV} \times {BMI}^{2} \times Age + {q}_{d} \times \overline{PEV} \times BMI \times {Age}^{2}$$

$$MAP = \frac{SBP + 2. \times DBP}{3.}$$

Constants aₛ−qₛ and a_{d}−q_{d} were calculated by using a curve fitting process where PWV's calculated on the device 100 were fit to the results taken from a statistically valid sample set of number known blood pressures (blood pressures taken from traditional means−cuff-based measuring)

The curve fitting process began by recording blood pressure from a testing pool to form the data set. Participants that were selected to participate in the testing pool had their blood pressures measured with three different devices; a manual cuff-based blood pressure machine, and two different digital cuff-based blood pressure machines. The participants also used prototypes of cuffless blood pressure monitor 100 so that values for PTT, PET, PTV, PEV, and HR could be collected. The values measures for HR were verified through the use of a finger pulse oximeter to ensure accuracy moving forward. Participants also engaged in brief exercise to raise their heart rates, and repeated the prior described process so that data for an active state could also be collected, in conjunction with data that was collected while the participants were in a resting state.

Once all of the data was collected, it was then uploaded into a curve fitting software and had a filter applied to it. This filter ensured that only accurate readings were used and also complied the data into a single set for the software to use during the curve fitting process. That final data set was then subjected to the curve fitting process so that the most accurate values for constants aₛ−qₛ and a_{d}−q_{d} were determined.

The end result of the curve fitting process allows cuffless blood pressure monitor 100 to be more accurate and stable than current digital cuff-based blood pressure monitors available to consumers. Currently, the systolic and diastolic blood pressures calculated with the cuffless blood pressure monitor 100 has a Mean Absolute Error (MAE) of less than 5 mmHg when compared to readings taken with the industry approved manual cuff-based measurement device.

In the current embodiment, cuffless blood pressure monitor 100 will have MAP values preloaded onto the device so that it can compare a user's readings to those preloaded values in order to make a determination as to whether the calculated MAP value is low, high, or normal. Cuffless blood pressure monitor 100 will then be able to display the results of the comparison on display 104 so that a user can see whether their blood pressure is within a healthy range. In the present embodiment, the MAP result will be displayed, along with the calculated SBP, DBP, and heart rate.

Referring now to FIG. 6, a systems level diagram of cuffless blood pressure monitor 100 is shown. Shown on the right of the diagram, are the three different components that make up control system 400. Control system 400 consists of microcontroller unit 402, Wi-Fi/Bluetooth module 406, and analog-to-digital converter (ADC) 404. Microcontroller unit 402 will be preprogrammed to perform all of the equations listed during the discussion of FIG. 5. ADC 404 will be responsible for converting the recorded ECG signal 302 and PPG signal 304 into a medium that allows microcontroller unit 402 to perform the required calculations. Wi-Fi/Bluetooth module 406 will make it so that cuffless blood pressure monitor 100 can wirelessly communicate with either a computer, smart phone, or smart tablet.

In the present embodiment, cuffless blood pressure monitor 100 is powered by battery 408 which distributes power to the entire unit through power regulator 410. Power regulator 410 will ensure that each of the different components installed on cuffless blood pressure monitor 100 receives the requisite power supply without damaging the component. In the present embodiment, battery 408 is a lithium-ion polymer battery; however, other battery types currently available are fully envisioned.

To the left of the diagram is ECG filter 412, PPG filter 414, and temperature sensor 416. Both ECG filter 412 and PPG filter 414 play a crucial role in ensuring that accurate readings are eventually used by micro controller unit 402 when calculating a user's blood pressure and heart rate. Temperature sensor 416 senses a user's temperature and can determine whether a fever is present.

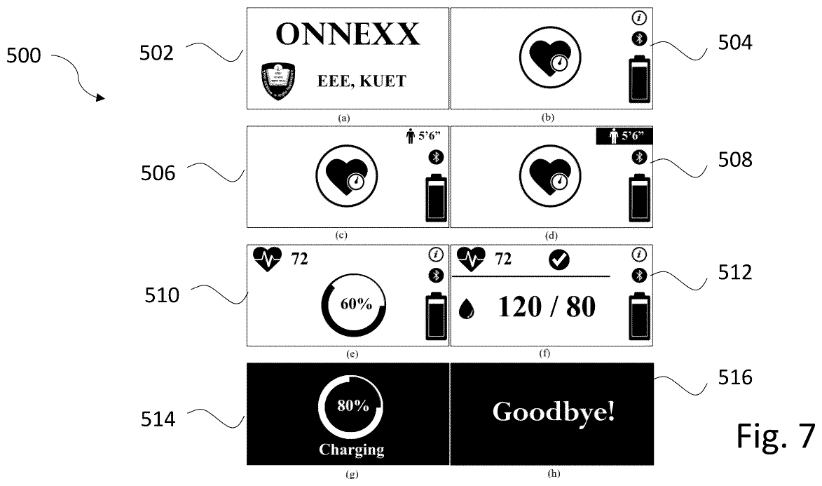

Referring now to FIG. 7, a prototype of user interface for cuffless blood pressure monitor 100 is shown. Generally, the user interface for cuffless blood pressure monitor 100 is designated as system 500 and FIG. 7 shows the different screens that system 500 may display. None of these figures are intended to be limiting, and instead are shown to merely provide an illustrative example of a possible user interface for cuffless blood pressure monitor 100.

The first image is bootup screen 502 which will be displayed once the user turns on the device. Home screen 504 shows the battery percentage of the device, as well as the status of a possible Bluetooth connection. Home screen 504 here only shows the battery percentage and Bluetooth connection status because no user has entered in any information about their height, nor has a user started using cuffless blood pressure monitor to track their heart rate and blood pressure.

Screens 506 and 508 show the display for system 500 when the user height is finally selected. For screen 506 the user height is bolded because the user in this stage is still selecting the correct height. However, screen 508 shows the user height not bolded which means that the height is finalized and stored within the microcontroller of cuffless blood pressure monitor 100. Screens 510 and 512 show the different possible displays of system 500 when cuffless blood pressure monitor 100 is in use. For screen 510, only the heart rate of the user is being tracked, but once the relevant calculations are performed a user's blood pressure will be displayed.

If the device is being charged, then it is possible for screen 514 to be displayed to show the state of charge for the battery. This makes it easy for a user to determine how quickly the device is charging, and to also determine how much charge they need cuffless blood pressure monitor 100 to have throughout the day. Finally, screen 516 shows a display that system 500 may show when the device is being powered off.

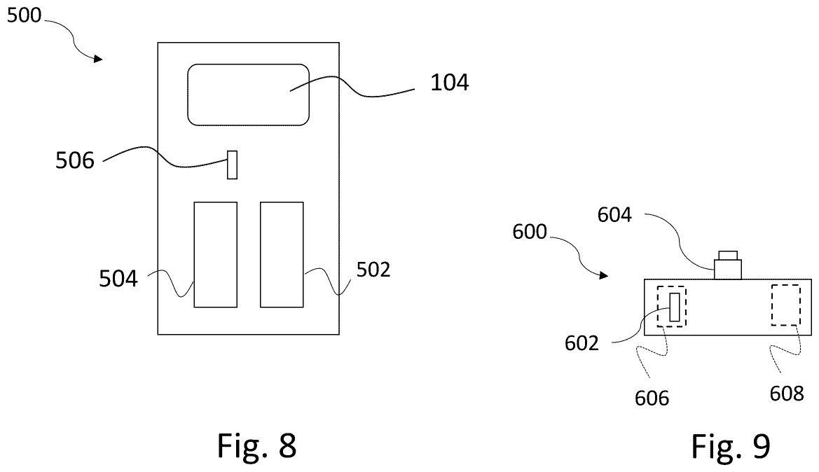

Referring now to FIG. 8, an alternative embodiment of the present invention is shown. In this embodiment, the cuffless blood pressure monitor 100 will be installed onto a smart scale 500. Smart scale 500 will have the same firmware as the handheld embodiment of cuffless blood pressure monitor 100.

The difference will be in how the required measurements to perform the prior mentioned calculations are done. Smart scale 500 measures the ECG signal and PPG signal from a user's feet instead of from their hand as described earlier. Right scale electrode 502 and left scale electrode 504 will be installed so that a user can comfortably stand on smart scale 500 while their ECG signal is measured. Further, scale PPG sensor 506 will be installed just above left scale electrode 504 so that the PPG signal can be measured from a user's left hallux (big toe).

Referring now to FIG. 9, an alternative embodiment of the present invention is shown and generally designated 600. In this embodiment, the cuffless blood pressure monitor 600 is plugged directly into a smart phone or tablet (not shown) where the readings will be displayed. This embodiment will be highly compact and easier to transport the system from location to location. Connection 604 is a USB-connection sized for connection into commonly sold smartphones. There will be Left Electrode 606 and Right Electrode 608 (both shown in dashed lines) installed onto the back of the device, and PPG Sensor 602 will be installed on the front surface to contact the user's thumb as described above.

While the cuffless blood pressure monitor 100 of the present invention as herein shown and disclosed in detail is fully capable of obtaining the objects and providing the advantages herein before stated, it is to be understood that it is merely illustrative of preferred and alternative embodiments of the invention and that no limitations are intended to the details of construction or design herein shown other than as described in the appended claims.

I claim:

1. A cuffless blood pressure monitor comprising:
a left electrode;
a right electrode;
a PPG sensor;
a control system configured to collect readings from the left electrode, the right electrode, and the PPG sensor, and
wherein the control system is further configured to calculate a user's blood pressure based on readings that are collected from the left electrode, the right electrode, and the PPG sensor.

2. The cuffless blood pressure monitor of claim 1, further comprising a display that is configured to show the calculated blood pressure.

3. The cuffless blood pressure monitor of claim 2, wherein the control system further comprises a microcontroller, an analog-to-digital converter, a wireless communication module, and a battery back.

4. The cuffless blood pressure monitor of claim 3, wherein the blood pressure displayed is a calculated mean arterial pressure that is based on a calculated systolic pressure and a calculated diastolic blood pressure.

5. The cuffless blood pressure monitor blood pressure monitor of claim 4, wherein the display is further configured to show the calculated mean arterial pressure, the calculated systolic blood pressure, and the diastolic blood pressure.

6. The cuffless blood pressure monitor of claim 5, wherein the control system further comprises of preloaded mean arterial pressures that are compared to the calculated mean arterial pressure by the control system so that the control system can determine whether the calculated mean arterial pressure is low, normal, or high.

7. The cuffless blood pressure monitor of claim 6, wherein the display is further configured to show whether the calculated mean arterial pressure is low, normal, or high.

8. The cuffless blood pressure monitor of claim 7, wherein the display is further configured to show a heart rate of a user.

9. A cuffless blood pressure monitor comprising:
a display
a left electrode;
a right electrode;
a PPG sensor;
a control system that is in electrical communication with the display, the left electrode, the right electrode, and the PPG sensor, and
wherein the control system is further configured to collect readings from the left electrode, the right electrode, and the PPG sensor in order calculate a user's blood pressured based on readings that are collected from the left electrode, the right electrode, and the PPG sensor.

10. The cuffless blood pressure monitor of claim 9, wherein the control system further comprises a microcontroller, an analog-to-digital converter, a wireless communication module, and a battery back.

11. The cuffless blood pressure monitor of claim 10, wherein the blood pressure displayed is a calculated mean arterial pressure that is based on a calculated systolic pressure and a calculated diastolic blood pressure.

12. The cuffless blood pressure monitor blood pressure monitor of claim 11, wherein the display is further configured to display the mean arterial pressure, the calculated systolic blood pressure, and the diastolic blood pressure.

13. The cuffless blood pressure monitor of claim 12 wherein the control system further comprises of preloaded mean arterial pressures that are compared to the calculated mean arterial pressure by the control system so that the control system can determine whether the calculated mean arterial pressure is low, normal, or high.

14. The cuffless blood pressure monitor of claim 13, wherein the display is further configured to display whether the calculated mean arterial pressure is low, normal, or high.

15. The cuffless blood pressure monitor of claim 7 wherein the display is further configured to show a heart rate of a user.

16. A cuffless blood pressure monitor comprising:
a left electrode, a right electrode, and a PPG sensor that are in electrical communication with a control system that is configured to collect readings from the left electrode, the right electrode, and the PPG sensor,
wherein the control system is configured to calculate a systolic blood pressure, a diastolic blood pressure, and to calculate a mean arterial pressure that is based on the calculated systolic blood pressure and the calculated diastolic blood pressure; and
a display that is configured to display the calculated mean arterial blood pressure, the calculated systolic blood pressure, and the calculated diastolic blood pressure.

17. The cuffless blood pressure monitor of claim 16, wherein the control system further comprises a microcontroller, an analog-to-digital converter, a wireless communication module, and a battery back.

18. The cuffless blood pressure monitor of claim 17 wherein the control system has preloaded mean arterial pressures to compare to the calculated mean arterial pressure to determine whether the mean arterial pressure is low, normal, or high.

19. The cuffless blood pressure monitor of claim 18, wherein the display is further configured to show whether the calculated mean arterial pressure is low, normal, or high.

20. The cuffless blood pressure monitor of claim 19, wherein the display is further configured to show a heart rate of a user.
